(19) United States
(12) Patent Application Publication
(10) Pub. No.: US 2005/0004094 A1
(43) Pub. Date: Jan. 6, 2005

(54) Cephem compounds

(75) Inventors: Toshio Yamanaka, Osaka (JP); Hidenori Ohki, Osaka (JP); Masaru Ohgaki, Osaka (JP); Shinya Okuda, Osaka (JP); Ayako Toda, Osaka (JP); Kohji Kawabata, Osaka (JP); Satoshi Inoue, Hiroshima (JP); Keiji Misumi, Hiroshima (JP); Kenji Itoh, Hiroshima (JP); Kenji Satoh, Hiroshima (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Fujisawa Pharmaceutical Co., Ltd., Osaka-shi (JP); WAKUNAGA PHARMACEUTICAL CO., LTD., Osaka-shi (JP)

(21) Appl. No.: 10/833,088

(22) Filed: Apr. 28, 2004

(30) Foreign Application Priority Data
May 16, 2003 (AU) 2003902380

(51) Int. Cl. A61K 31/545; C07D 501/14

(52) U.S. Cl. 514/202; 540/222

(57) ABSTRACT

The present invention relates to a compound of the formula [I]: 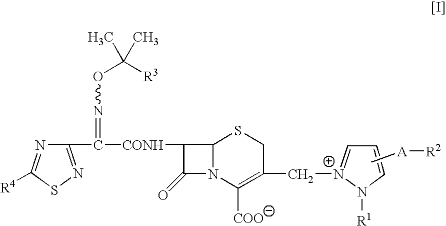 wherein
R¹ is lower alkyl which may have suitable substituent(s),
R² is amino, protected amino or guanidino,
R³ is carboxy or protected carboxy,
R⁴ is amino or protected amino, and A is lower alkylene, or a pharmaceutically acceptable salt thereof, a process for preparing a compound of the formula [I], and a pharmaceutical composition comprising a compound of the formula [I] in admixture with a pharmaceutically acceptable carrier.

### TECHNICAL FIELD

The present invention relates to new cephem compounds and pharmaceutically acceptable salts thereof. More particularly, the present invention relates to new cephem compounds and pharmaceutically acceptable salts thereof, which have antimicrobial activities, to processes for preparation thereof, to pharmaceutical composition comprising the same, and to a method for treating infectious diseases in human being and animals.

### DISCLOSURE OF INVENTION

One object of the present invention is to provide novel cephem compounds and pharmaceutically acceptable salts thereof, which are highly active against a number of pathogenic microorganisms.

Another object of the present invention is to provide processes for the preparation of said cephem compounds and salts thereof.

A further object of the present invention is to provide a pharmaceutical composition comprising, as an active ingredient, said cephem compounds or their pharmaceutically acceptable salts.

Still further object of the present invention is to provide a method for treating infectious diseases caused by pathogenic microorganisms, which comprises administering said cephem compounds to infected human being or animals.

The object cephem compounds of the present invention are novel and can be represented by the following general formula [I]: 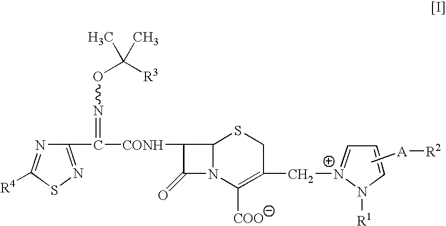
wherein
R¹ is lower alkyl which may have suitable substituent(s),
R² is amino, protected amino or guanidino,
R³ is carboxy or protected carboxy,
R⁴ is amino or protected amino, and
A is lower alkylene.

As to the object compound [I], the following points are to be noted.

That is, the object compound [I] includes syn isomer (Z form), anti isomer (E form) and a mixture thereof. Syn isomer (Z form) means one geometrical isomer having the partial structure represented by the following formula: 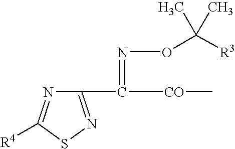
wherein R³ and R⁴ are each as defined above, and anti isomer (E form) means the other geometrical isomer having the partial structure represented by the following formula: 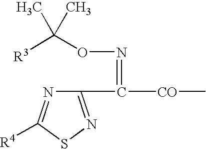
wherein R³ and R⁴ are each as defined above, and all of such geometrical isomers and mixture thereof are included within the scope of this invention.

In the present specification and claims, the partial structure of these geometrical isomers and mixture thereof are represented for convenience' sake by the following formula: 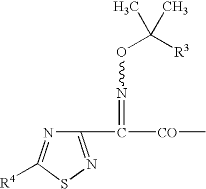
wherein R³ and R⁴ are each as defined above.

Another point to be noted is that the pyrazolio moiety of the compound [I] can also exist in the tautomeric form, and such tautomeric equilibrium can be represented by the following formula. 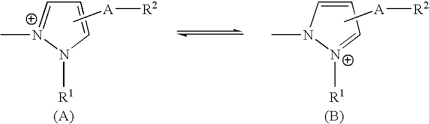
wherein R¹, R² and A are each as defined above.

Both of the above tautomeric isomers are included within the scope of the present invention, and in the present specification and claims, however, the object compound [I] is represented for convenience' sake by one expression of the pyrazolio group of the formula (A).

The cephem compound [I] of the present invention can be prepared by the following processes as illustrated in the following. 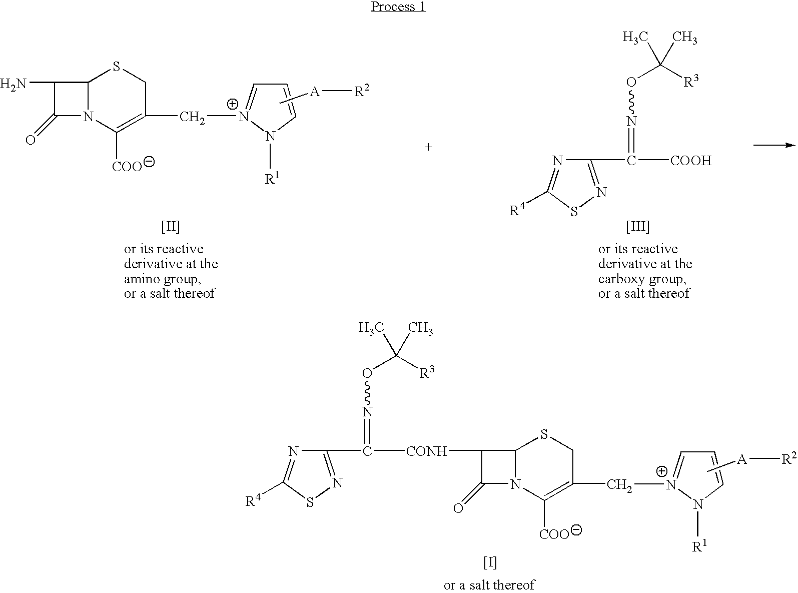 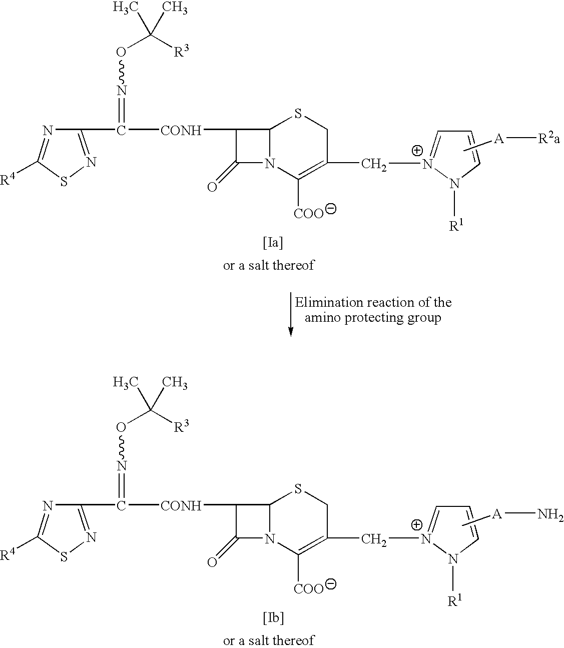 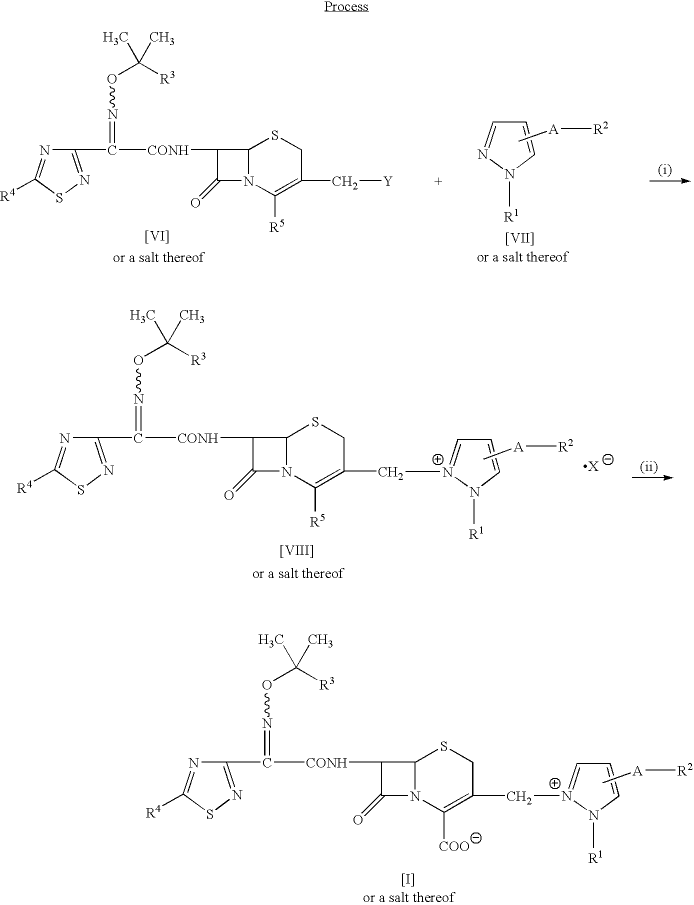
wherein R¹, R², R³, R⁴ and A are each as defined above,
R²a is protected amino,
R⁵ is protected carboxy,
Y is a leaving group, and
X^{\mathring{-}} is an anion.

The starting compounds [II] and [VI] can be prepared by the following processes. 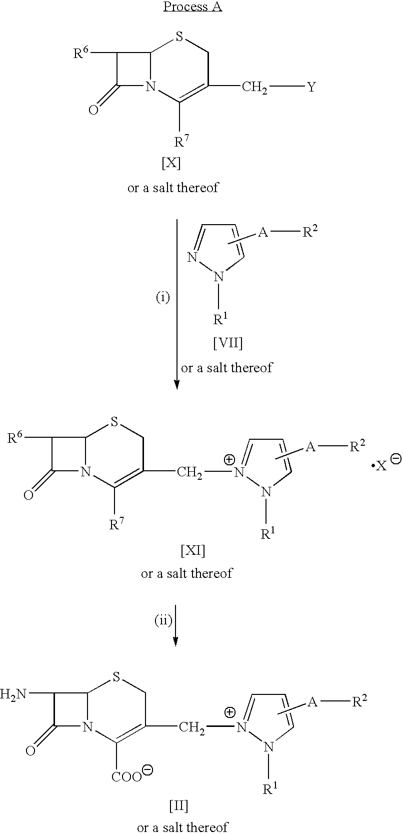 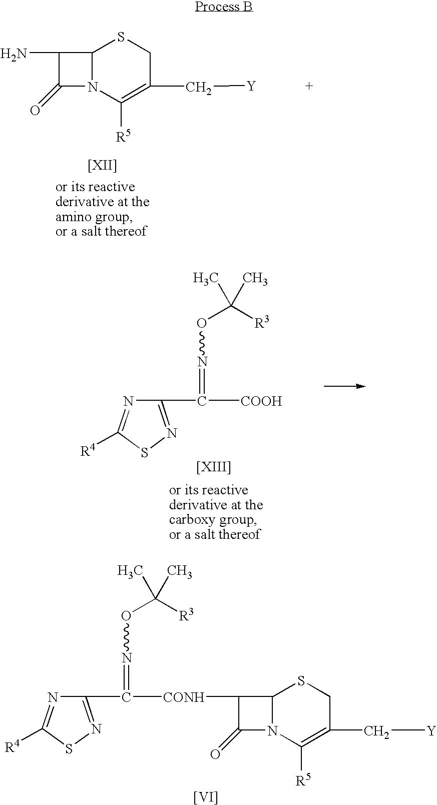
wherein R¹, R², R³, R⁴, A, R⁵, Y and X^{\mathring{-}} are each as defined above,
R⁶ is protected amino, and
R⁷ is protected carboxy.

The starting compound [VII] or salts thereof can be prepared by the methods disclosed in the Preparations 1-6 and 8-13 described later or similar manners thereto.

In the above and subsequent descriptions of this specification, suitable examples of the various definitions are explained in detail as follows.

The term “lower” is used to mean a group having 1 to 6, preferably 1 to 4, carbon atom(s), unless otherwise indicated.

Suitable “lower alkyl” includes straight or branched alkyl having 1 to 6 carbon atom(s), such as methyl, ethyl, propyl, isopropyl, butyl, isobutyl, sec-butyl, tert-butyl, pentyl, tert-pentyl and hexyl, in which more preferred one is C₁-C₄ alkyl.

Suitable examples of “substituent(s)” in “lower alkyl which may have suitable substituent(s)” may include one or more (preferably 1 to 3) substituent(s) selected from amino, hydroxy, halogen, cyano, C₁-C₆ alkoxy, C₁-C₆ alkylthib, aryl(C₁-C₆)alkyloxy, carboxy, and the like.

Suitable “lower alkylene” includes straight or branched alkylene having 1 to 6 carbon atoms, such as methylene, ethylene, trimethylene, tetramethylene, pentamethylene, hexamethylene and propylene, in which more preferred one is straightalkylene having 1 to 3 carbon atoms, and the most preferred one is ethylene.

Suitable “amino protecting group” in “protected amino” includes an acyl group as mentioned below, substituted or unsubstituted aryl (lower) alkylidene [e.g., benzylidene, hydroxybenzylidene, etc.], aryl (lower) alkyl such as mono-, di- or triphenyl (lower) alkyl [e.g., benzyl, phenethyl, benzhydryl, trityl, etc.], and the like.

Suitable “acyl” includes lower alkanoyl [e.g., formyl, acetyl, propionyl, hexanoyl, pivaloyl, etc.], mono(or di or tri) halo (lower) alkanoyl [e.g., chloroacetyl, trifluoroacetyl, etc.], lower alkoxycarbonyl [e.g., methoxycarbonyl, ethoxycarbonyl, tert-butoxycarbonyl, tert-pentyloxycarbonyl, hexyloxycarbonyl, etc.], carbamoyl, aroyl [e.g., benzoyl, toluoyl, naphthoyl, etc.], aryl(lower)alkanoyl [e.g., phenylacetyl, phenylpropionyl, etc.], aryloxycarbonyl [e.g., phenoxycarbonyl, naphthyloxycarbonyl, etc.], aryloxy (lower) alkanoyl [e.g., phenoxyacetyl, phenoxypropionyl, etc.], arylglyoxyloyl [e.g., phenylglyoxyloyl, naphthylglyoxyloyl, etc.], aryl (lower) alkoxycarbonyl which optionally substituted by suitable substituent(s) [e.g., benzyloxycarbonyl, phenethyloxycarbonyl, p-nitrobenzyloxycarbonyl, etc.], and the like.

Preferable examples of the protected amino group include aryl(lower)alkylamino and lower alkanoylamino, in which more preferred ones are triphenyl (C₁-C₄) alkylamino and C₁-C₄ alkanoylamino, and the particularly preferred ones are tritylamino and formylamino.

Suitable “protected carboxy” includes an esterified carboxy group and the like, and concrete examples of the ester moiety in said esterified carboxy group include the ones such as lower alkyl ester [e.g., methyl ester, ethyl ester, propyl ester, isopropyl ester, butyl ester, isobutyl ester, tert-butyl ester, pentyl ester, hexyl ester, 1-cyclopropylethyl ester, etc.] which may have suitable substituent(s), for example, lower alkanoyloxy (lower)alkyl ester [e.g., acetoxymethyl ester, propionyloxymethyl ester, butyryloxymethyl ester, valeryloxymethyl ester, pivaloyloxymethyl ester, 1-acetoxyethyl ester, 1-propionyloxyethyl ester, 2-propionyloxyethyl ester, hexanoyloxymethyl ester, etc.], lower alkanesulfonyl (lower) alkyl ester [e.g., 2-mesylethyl ester, etc.] or mono(or di or tri)halo(lower)alkyl ester [e.g., 2-iodoethyl ester, 2,2,2-trichloroethyl ester, etc.]; lower alkenyl ester [e.g., vinyl ester, allyl ester, etc.]; lower alkynyl ester [e.g., ethynyl ester, propynyl ester, etc.]; aryl(lower)alkyl ester which may have suitable substituent(s) [e.g., benzyl ester, 4-methoxybenzyl ester, 4-nitrobezyl ester, phenethyl ester, trityl ester, benzhydryl ester, bis(methoxyphenyl)methyl ester, 3,4-dimethoxybenzyl ester, 4-hydroxy-3,5-di-tert-butylbenzyl ester, etc.]; aryl ester which may have suitable substituent(s) [e.g., phenyl ester, 4-chlorophenyl ester, tolyl ester, 4-tert-butylphenyl ester, xylyl ester, mesityl ester, cumenyl ester, .etc.];.,and the like, in which the preferred ones are C₁-C₄-alkyl ester and mono or di or triphenyl(C₁-C₄)alkyl ester, and the particularly preferred ones are tert-butyl ester and benzhydryl ester.

Suitable “leaving group” includes halogen [e.g., chlorine, bromine, iodine, etc.] or acyloxy such as arylsulfonyloxy [e.g., benzenesulfonyloxy, tosyloxy, etc.], lower alkylsulfonyloxy [e.g., mesyloxy, etc.], lower alkanoyloxy [e.g., acetyloxy, propionyloxy, etc.], and the like.

Suitable “anion” includes formate, acetate, trifluoroacetate, maleate, tartrate, methanesulfonate, benzenesulfonate, toluenesulfonate, chloride, bromide, iodide, sulfate, hydrogensulfate, phosphate, and the like.

Suitable pharmaceutically acceptable salts of the object compound [I] are conventional non-toxic salts and include, for example, a salt with a base or an acid addition salt such as a salt with an inorganic base, for example, an alkali metal salt [e.g., sodium salt, potassium salt, etc.], an alkaline earth metal salt [e.g., calcium salt, magnesium salt, etc.], an ammonium salt; a salt with an organic base, for example, an organic amine salt [e.g., trimethylamine salt, triethylamine salt, pyridine salt, picoline salt, ethanolamine salt, triethanolamine salt, dicyclohexylamine salt, N,N′-dibenzylethylenediamine salt, etc.]; an inorganic acid addition salt [e.g., hydrochloride, hydrobromide, sulfate, hydrogensulfate, phosphate, etc.]; an organic carboxylic or sulfonic acid addition salt [e.g., formate, acetate, trifluoroacetate, maleate, tartrate, citrate, fumarate, methanesulfonate, benzenesulfonate, toluenesulfonate, etc.]; and a salt with a basic or acidic amino acid [e.g., arginine, aspartic acid, glutamic acid, etc.].

The preferred embodiment of the cephem compound of the present invention represented by the general formula [I] is as follows.

The compound of the formula [I] wherein
R¹ is (C₁-C₆)alkyl,
R² is amino or guanidino,
R³ is carboxy,
R⁴ is amino, and
A is (C₁-C₆)alkylene,
   or a pharmaceutically acceptable salt thereof.

The processes for preparing the object compound of the present invention are explained in detail in the following.

Process 1

The compound [I] or a salt thereof can be prepared by reacting the compound [II] or its reactive derivative at the amino group, or a salt thereof with the compound [III] or its reactive derivative at the carboxy group, or a salt thereof.

Suitable reactive derivative at the amino group of the compound [II] includes Schiff's base type imino or its tautomeric enamine type isomer formed by the reaction of the compound [II] with a carbonyl compound such as aldehyde, ketone and the like; a silyl derivative formed by the reaction of the compound [II] with a silyl compound such as bis(trimethylsilyl)acetamide, mono(trimethylsilyl)acetamide [e.g., N-(trimethylsilyl)acetamide], bis(trimethylsilyl)urea and the like; a derivative formed by the reaction of the compound [II] with phosphorus trichloride or phosgene.

Suitable salts of the compound [II] and its reactive derivative can be-referred to the ones as exemplified for the compound [I].

Suitable reactive derivative at the carboxy group of the compound [III] includes an acid halide, an acid anhydride, an activated amide, and an activated ester. A suitable example of the reactive derivatives may be an acid chloride; an acid azide; a mixed acid anhydride with an acid such as substituted phosphoric acid [e.g., dialkylphosphoric acid, phenylphosphoric acid, diphenylphosphoric acid, dibenzylphdsphoric acid, halogenated phosphoric acid, etc.], dialkylphosphorous acid, sulfurous acid, thiosulfuric acid, sulfuric acid, alkanesulfonic acid [e.g., methanesulfonic acid, etc.], aliphatic carboxylic acid [e.g., acetic acid, propionic acid, butyric acid, isobutyric acid, pivalic acid, pentanoic acid, isopentanoic acid, 2-ethylbutyric acid, trichloroacetic acid, etc.] and aromatic carboxylic acid [e.g., benzoic acid, etc.]; a symmetrical acid anhydride; an activated amide with imidazole, 4-substituted imidazole, dimethylpyrazole, triazole or tetrazole; an activated ester [e.g., cyanomethyl ester, methoxymethyl ester, dimethyliminomethyl [(CH₃)₂N⁺═CH—] ester, vinyl ester, propargyl ester, p-nitrophenyl ester, 2,4-dinitrophenyl ester, trichlorophenyl ester, pentachlorophenyl ester, mesyiphenyl ester, phenylazophenyl ester, phenyl thioester, p-nitrophenyl thioester, p-cresyl thioester, carboxymethyl thioester, pyranyl ester, pyridyl ester, piperidyl ester, 8-quinolyl thioester, etc.]; or an ester with an N-hydroxy compound [e.g., N,N-dimethylhydroxylamine, 1-hydroxy-2-(1H)-pyridone, N-hydroxysuccinimide, N-hydroxyphthalimide, N-hydroxy-1H-benzotriazole, etc.]. These reactive derivatives can optionally be selected from them according to the kind of the compound [III] to be used.

Suitable salts of the compound [III] and its reactive derivative can be referred to the ones as exemplified for the, compound [I].

The reaction is usually carried out in a conventional solvent such as water, alcohol [e.g., methanol, ethanol, etc.], acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction. These conventional solvents may also be used in a mixture with water.

In this reaction, when the compound [III] is used in free acid form or its salt form, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl) carbodiimide; N,N′-diethylcarbodiimide; N,N′-diisopropylcarbodiimide; N-ethyl-N′-(3-dimethylaminopropyl)carbodiimide; N,N′-carbonyl-bis-(2-methylimidazole); pentamethyleneketene-N-cyclohexylimine; diphenylketene-N-cyclohexylimine; ethoxyacetylene; 1-alkoxy-1-chloroethylene; trialkyl phosphite; ethyl polyphosphate; isopropyl polyphosphate; phosphorus oxychloride (phosphoryl chloride); phosphorus trichloride; thionyl chloride; oxalyl chloride; lower alkyl haloformate [e.g., ethyl chloroformate, isopropyl chloroformate, etc.], triphenylphosphine; 2-ethyl-7-hydroxybenzisoxazolium salt; 2-ethyl-5-(m-sulfophenyl) isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, trichloromethyl chloroformate, phosphorus oxychloride, etc.; and the like.

The reaction may also be carried out in the presence of an inorganic or organic base such as an alkali metal bicarbonate, tri(lower)alkylamine, pyridine, N-(lower)alkylmorpholine, N,N7-di(lower)alkylbenzylamine, and the like.

The reaction temperature is not critical, and the reaction is usually carried out under cooling to warming.

Process 2

The compound [Ib] or a salt thereof can be prepared by subjecting the compound [Ia] or a salt thereof to elimination reaction of the amino protecting group.

Elimination reaction is carried out in accordance with a conventional method such as hydrolysis, reduction and the like.

The hydrolysis is preferably carried out in the presence of a base or an acid including Lewis acid.

Suitable base includes an inorganic base and an organic base such as an alkali metal [e.g., sodium, potassium, etc.], an alkaline earth metal [e.g., magnesium, calcium, etc.], the hydroxide or carbonate or hydrogen carbonate thereof, trialkylamine [e.g., trimethylamine, triethylamine, etc.], picoline, 1,5-diazabicyclo[4.3.0]non-5-ene, 1,4-diazabicyclo[2.2.2]octane, 1,8-diazabicyclo[5.4.0]undec-7-ene, and the like.

Suitable acid includes an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen chloride, hydrogen bromide, etc.].

The elimination using Lewis acid such as trihaloacetic acid [e.g., trichloroacetic acid, trifluoroacetic acid, etc.], and the like is preferably carried out in the presence of cation trapping agents [e.g., anisole, phenol, etc.].

The reaction is usually carried out in a solvent such as water, alcohol [e.g., methanol, ethanol, etc.], methylene chloride, tetrahydrofuran, a mixture thereof or any other solvent which does not adversely influence the reaction. A liquid base or acid can be also used as a solvent.

The reaction temperature is not critical and the reaction is usually carried out under cooling to warming.

The reaction method applicable for the elimination reaction may include chemical reduction and catalytic reduction.

Suitable reducing reagents to be used in chemical reduction are a combination of a metal [e.g., tin, zinc, iron, etc.] or metallic compound [e.g., chromium chloride, chromium acetate, etc.] and an organic acid or inorganic acid [e.g., formic acid, acetic acid, propionic acid, trifluoroacetic acid, p-toluenesulfonic acid, hydrochloric acid, hydrobromicacid, etc.].

Suitable catalysts to be used in catalytic reduction are conventional ones such as platinum catalysts [e.g., platinum plate, spongy platinum, platinum black, colloidal platinum, platinum oxide, platinum wire, etc.], palladium catalysts [e.g., spongy palladium, palladium black, palladium oxide, palladium on carbon, colloidal palladium, palladium on barium sulfate, palladium on barium carbonate, etc.], nickel catalysts [e.g., reduced nickel, nickel oxide, Raney nickel, etc.], cobalt catalysts [e.g., reduced cobalt, Raney cobalt, etc.], iron catalysts [e.g., reduced iron, Raney iron, etc.], copper catalysts [e.g., reduced copper, Raney copper, Ullman copper, etc.] and the like.

The reduction is usually carried out in a conventional solvent which does not adversely influence the reaction such as water, methanol, ethanol, propanol, N,N-dimethylformamide or a mixture thereof.

Additionally, in case that the above-mentioned acids to be used in chemical reduction are liquid, they can also be used as a solvent.

Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

Process 3-(i)

The compound [VIII] or a salt thereof can be prepared by reacting the compound [VI] or a salt thereof with the compound [VII] or a salt thereof.

Suitable salt of the compounds [VI], [VII] and [VIII] can be referred to the ones as exemplified for the compound [I].

The present reaction may be carried out in a solvent such as water, phosphate buffer, acetone, chloroform, acetonitrile, nitrobenzene, methylene chloride, ethylene chloride, formamide, N,N-dimethylformamide, methanol, ethanol, diethyl ether, tetrahydrofuran, dimethyl sulfoxide, or any other organic solvent which does not adversely affect the reaction, preferably in ones having strong polarities. Among the solvents, hydrophilic solvents may be used in a mixture with water. When the compound [VII] is liquid, it can also be used as a solvent.

The reaction is preferably conducted in the presence of a base, for example, an inorganic base such as alkali metal hydroxide, alkali metal carbonate, alkali metal hydrogen carbonate, an organic base such as trialkylamine, and the like.

The reaction temperature is not critical, and the reaction is usually carried out at ambient temperature, under warming or under heating. The present reaction is preferably carried out in the presence of alkali metal halide [e.g., sodium iodide, potassium iodide, etc.], alkali metal thiocyanate [e.g., sodium thiocyanate, potassium thiocyanate, etc.], and the like.

Anion X^{\mathring{-}} may be one derived from a leaving group Y, and it may be converted to other anion by a conventional method.

Process 3-(ii)

The compound [I] or a salt thereof can be prepared by subjecting the compound [VIII] or a salt thereof to elimination reaction of the carboxy protecting group.

Elimination reaction is carried out in a similar manner to the reaction in the aforementioned Process 2, and therefore the reagents to be used and reaction conditions (e.g., solvent, reaction temperature, etc.) can be referred to those of Process 2.

Processes A and B for the preparation of the starting compounds are explained in detail in the following.

Process A-(i)

The compound [XI] or a salt thereof can be prepared by reacting the compound [X] or a salt thereof with the compound [VII] or a salt thereof.

This reaction can be carried out in a similar manner to the reaction in the aforementioned Process 3-(i), and therefore the reagents to be used and reaction conditions (e.g., solvent, reaction temperature, etc.) can be referred to those of Process 3-(i).

Process A-(ii)

The compound [II] or a salt thereof can be prepared by subjecting the compound [XI] or a salt thereof to elimination reaction of the amino protecting groups in R⁶ and the carboxy protecting group in R⁷.

This reaction can be carried out in a similar manner to the reaction in the aforementioned Process 2, and therefore the reagents to be used and reaction conditions (e.g., solvent, reaction temperature, etc.) can be referred to those of Process 2.

Process B

The compound [VI] or a salt thereof can be prepared by reacting the compound [XII] or its reactive derivative at the amino group, or a salt thereof with the compound [XIII] or its reactive derivative at the carboxy group, or a salt thereof.

This reaction can be carried out in a similar manner to the reaction in the aforementioned Process 1, and therefore the reagents to be used and reaction conditions (e.g., solvent, reaction temperature, etc.) can be referred to those of Process 1.

The compounds obtained by the above processes can be isolated and purified by a conventional method such as pulverization, recrystallization, column chromatography, reprecipitation, and the like.

It is to be noted that the compound [I] and other compounds may include one or more stereoisomer(s) such as optical isomer(s) and geometrical isomer(s) due to asymmetric carbon atom(s) and double bond(s), and all of such isomers and mixtures thereof are included within the scope of this invention.

The object compounds [I] and pharmaceutically acceptable salts thereof include solvates [e.g., enclosure compounds (e.g., hydrate, etc.)].

The object compound [I] and pharmaceutically acceptable salts thereof are novel and exhibit high antimicrobial activity, inhibiting the growth of a wide variety of pathogenic microorganisms including Gram-positive and Gram-negative microorganisms and are useful as antimicrobial agents.

Now in order to show the utility of the object compound [I], the test data on MIC (minimal inhibitory concentration) of a representative compound of this invention are shown in the following.

Test Method:

In vitro antibacterial activity was determined by the two-fold agar-plate dilution method as described below.

One loopful of an overnight culture of each test strain in Trypticase-soy broth (10⁶ viable cells per ml) was streaked on heart infusion agar (HI-agar) containing graded concentrations of representative test compound, and the minimal inhibitory concentration (MIC) was expressed in μg/ml after incubation at 37° C. for 20 hours.

Test Compound

Compound (a): 3-[3-(2-aminoethyl)-2-methyl-1-pyrazolio]methyl-7β-[(Z)-2-(5-amino-1,2,4-thiadiazol-3-yl)-2-(1-carboxy-1-methylethoxyimino)acetamido]-3-cephem-4-carboxylic acid hydrogen sulfate (Example 1)

Compound (b): 3-{[3-(2-{[amino(imino)methyl]amino}ethyl)-2-methyl-1-pyrazolio]methyl}-7β-({(2Z)-2-(5-amino-1,2,4-thiadiazol-3-yl)-2-[(1-carboxy-1-methylethoxy)imino]ethanoyl}-amino)-3-cephem-4-carboxylate (Example 2)

Compound (c): 3-{[4-(2-aminoethyl)-2-methyl-1-pyrazolio]methyl}-7β-({(2Z)-2-(5-amino-1,2,4-thiadiazol-3-yl)-2-[(l-carboxy-1-methylethoxy)imino]ethanoyl}amino)-3-cephem-4-carboxylate (Example 3) Ceftazidime

Test Results:

TABLE 1

| Test strain | Test compound | MIC (μg/ml) |
|---|---|---|
| Pseudomonas aeruginosa | (a) | 1 |
| FP 1456 | (b) | 2 |
| | (c) | 2 |
| | Ceftazidime | 32 |

For therapeutic administration, the object compound [I] and pharmaceutically acceptable salts thereof of the present invention are used in the form of a conventional pharmaceutical preparation which contains said compound as an active ingredient, in admixture with pharmaceutically acceptable carriers such as an organic or inorganic solid or liquid excipient which is suitable for oral, parenteral or external administration. The pharmaceutical preparations may be in a solid form such as tablet, granule, powder, capsule, or in a liquid form such as solution, suspension, syrup, emulsion, lemonade and the like.

If needed, there may be included in the above preparations auxiliary substances, stabilizing agents, wetting agents and other commonly used additives such as lactose, citric acid, tartaric acid, stearic acid, magnesium stearate, terra alba, sucrose, corn starch, talc, gelatin, agar, pectin, peanut oil, olive oil, cacao butter, ethylene glycol, and the like.

While the dosage of the compound [I] may very from and also depend upon the age, conditions of the patient, a kind of diseases, a kind of the compound [I] to be applied, etc. In general amounts between 1 mg and 4,000 mg or even more per day may be administered to a patient. An average single dose of about 50 mg, 100 mg, 250 mg, 500 mg, 1000 mg or 2000 mg of the object compounds [I] of the present invention may be used in treating diseases infected by pathogenic microorganisms.

The following Preparations and Examples are given for the purpose of illustrating the present invention in more detail.

Preparation 1

To a solution of triethyl phosphonoacetate (26.9 g) in tetrahydrofuran (200 ml) was added sodium hydride (60% dispersion in mineral oil, 4.8 g) by portions under ice-cooling. The mixture was stirred at the same temperature for 1 hour. To the reaction mixture was added 1-methyl-1H-pyrazole-5-carbaldehyde (33.0 g) in tetrahydrofuran (165 ml) at room temperature, and the mixture was stirred at the same temperature for 1.5 hours. To the resulting solution was added 10% aqueous potassium hydrogen sulfate solution. The mixture was extracted with ethyl acetate twice. The combined organic layers were washed with saturated sodium hydrogen carbonate solution and brine. The extract was dried over anhydrous magnesium sulfate, filtered and concentrated in vacuo. The residue was purified by silica gel column chromatography to give ethyl (2E)-3-(1-methyl-1H-pyrazol-5-yl)-2-propenoate (33.4 g) as an oil.

IR (neat) 2981, 2943, 1713, 1703, 1180, 781 cm⁻¹.

¹H-NMR(DMSO-d₆) δ 1.26 (3H, t, J=7.1 Hz), 3.88 (3H, s), 4.20 (2H, q, J=7.1 Hz), 6.54 (1H, d, J=15.8 Hz), 6.88 (1H, d, J=1.8 Hz), 7.45 (1H, d, J=1.8 Hz), 7.60 (1H, d, J=15.8 Hz). MS (APCI) 181(M+H⁺).

Preparation 2

A solution of ethyl (2E)-3-(1-methyl-1H-pyrazol-5-yl)-2-propenoate (33.4 g) in ethanol (500 ml) was treated with 10% palladium on carbon (6.6 g) under a hydrogen atmosphere at room temperature for 2 hours. After the catalyst was filtered off, the filtrate was concentrated in vacuo togive ethyl 3-(1-methyl-1H-pyrazol-5-yl)propanoate (33.0 g) as a colorless oil.

IR(neat) 2981, 2941, 1733, 1541, 1398, 1182 cm⁻¹ .

¹H-NMR(DMSO-d₆) δ 1.17 (3H, t, J=7.1 Hz), 2.64 (2H, t, J=7.3 Hz), 2.86 (2H, t, J=7.3 Hz), 3.33 (3H, s), 4.07 (2H, q, J=7.1 Hz), 6.01 (1H, d, J=1.7 Hz), 7.26 (1H, d, J=1.7 Hz).

MS(APCI) 183 (M+H⁺).

Preparation 3

To a solution of ethyl 3-(1-methyl-1H-pyrazol-5-yl)propanoate (33 g) in ethanol (330 ml) was added 1.0N aqueous sodium hydroxide solution (362 ml), and the mixture was stirred at 70° C. for 1 hour. After cooling to room temperature, ethanol was evaporated. The solution was extracted with diethyl ether. The aqueous layer was acidified to pH 3 with 1.0N aqueous hydrochloric acid solution and the mixture was extracted with ethyl acetate four times. The extract was dried over anhydrous magnesium sulfate, filtered and concentrated in vacuo. The residue was triturated with diisopropyl ether and dried in vacuo to give 3-(1-methyl-1H-pyrazol-5-yl)propanoic acid (20.1 g) as a colorless solid.

IR (KBr) 1718, 1302, 1190, 1009, 945, 802, 633 cm⁻¹.

¹H-NMR (DMSO-d₆) δ 2.56 (2H, t, J=7.7 Hz), 2.82 (2H, t, J=7.7 Hz), 3.73 (3H, s), 6.01 (1H, d, J=1.7 Hz), 7.26 (1H, d, J=1.7 Hz), 11.90-12.70 (1H, br).

MS(APCI) 155 (M+H⁺).

Preparation 4

To a solution of 3-(1-methyl-1H-pyrazol-5-yl)propanoic acid (10.0 g) in 2-methyl-2-propanol (200 ml) were added diphenylphosphoryl azide (16.8. ml) and triethylamine (10.8 ml). The mixture was refluxed for 7 hours. After cooling to room temperature, ethyl acetate was added to the reaction mixture, and the mixture was washed with saturated aqueous sodium hydrogen carbonate, water and brine. The extract was dried over anhydrous magnesium sulfate, filtered and concentrated in vacuo. The residue was purified by silica gel column chromatography to give tert-butyl 2-(1-methyl-1H-pyrazol-5-yl)ethylcarbamate (12.6 g) as a solid.

IR (KBr) 1701, 1541, 1275, 1250, 777 cm⁻¹.

¹H-NMR(DMSO-d₆) δ 1.37 (9H, s), 2.72 (2H, t, J=7.0 Hz), 3.15 (2H, dt, J=5.5, 7.0 Hz), 3.71 (3H, s), 6.04 (1H, d, J=1.7 Hz), 6.96 (1H, t, J=5.5 Hz), 7.27 (1H, d, J=1.7 Hz).

MS (ESI) 226.4 (M+H⁺).

### EXAMPLE 1

To a solution of benzhydryl 7β-[(Z)-2-(5-amino-1,2,4-thiadiazol-3-yl)-2-(1-tert-butoxycarbonyl-1-methylethoxyimino)acetamido]-3-iodomethyl-3-cephem-4-carboxylate (2.5 g) in N,N-dimethylformamide (5.0 L) wasadded N-(trimethylsilyl) acetamide (2.26 g). After stirring at room temperature for 0.5 hour, tert-butyl 2-(1-methyl-1H-pyrazol-5-yl)ethylcarbamate (968 mg) was added to the mixture. Stirring was continued at 37° C. for 24 hours. The resulting mixture was poured into water and extracted with ethyl acetate. The organic layer was washed with water, 10% aqueous sodium thiosulfate, brine and 10% aqueous sodium trifluoroacetate, dried over anhydrous magnesium sulfate, filtered and evaporated in vacuo. The residue was dissolved in small amount of ethyl acetate, and the solution was added to diisopropyl ether dropwise. The precipitate was :filtered.. The filter cake was washed with diisopropyl ether and dried under vacuum.

To a solution of the resulting solid in methylene chloride (3.6 ml) were added anisole (1.8 ml) and trifluoroacetic acid (5.4 ml). The resulting solution was stirred at room temperature for 4 hours and poured into diisopropyl ether. The resulting precipitate was collected by filtration and dried in vacuo.

The crude product was purified by preparative high-performance liquid chromatography (HPLC) utilizing ODS column. The first eluate containing a desired product was concentrated to about 30 ml in vacuo. The concentrate was adjusted to about pH 3 by addition of concentrated hydrochloric acid and chromatographed on Diaion® HP-20 (Mitsubishi Chemical Corporation) eluting with 30% aqueous 2-propanol. The eluate was concentrated to about 30 ml in vacuo, added with 2.0M aqueous sulfuric acid (0.080 ml) and lyophilized to give 3-[3-(2-aminoethyl)-2-methyl-1-pyrazolio]methyl-7β-[(Z)-2-(5-amino-1,2,4-thiadiazol-3-yl)-2-(1-carboxy-1-methylethoxyimino)acetamido]-3-cephem-4-carboxylic acid hydrogen sulfate (90 mg).

IR(KBr) 1781, 1716, 1676, 1633, 1153 cm⁻¹.

¹H-NMR (D₂O) δ 1.61 (6H, s), 3.26 and 3.56 (2H, ABq, J=17.9 Hz), 3.27 (2H, t, J=7.4 Hz), 3.43 (2H, t, J=7.4 Hz), 4.01 (3H, s), 5.29 (1H, d, J=4.8 Hz), 5.33 and 5.46 (2H, ABq, J=15.5 Hz), 5.90 (1H, d, J=4.8 Hz), 6.79 (1H, d, J=3.1 Hz), 8.21 (1H, d, J=3.1 Hz).
   MS(ESI) 594.2 (M+H⁺).
   Preparation 5

To a solution of tert-butyl 2-(1-methyl-1H-pyrazol-5-yl)ethylcarbamate (12.6 g) in 1,4-dioxane (126 ml) was added 4N hydrogen chloride in 1,4-dioxane (140 ml) dropwise under ice-cooling. The mixture was stirred at room temperature for 1.5 hours. The solvent was evaporated in vacuo and the residue was triturated with diethyl ether. The resulting solid was collected by filtration, washed with diethyl ether and dried under vacuum to give 2-(1-methyl-1H-pyrazbl-5-yl)ethanamine dihydrochloride (10.2 g).

IR (KBr) 1625, 802 cm⁻¹.

¹H-NMR (DMSO-d₆) δ 2.90-3.15 (4H, m), 3.82 (3H, s), 6.28 (1H, d, J=2.0 Hz), 7.52 (1H, t, J=2.0 Hz), 7.60-8.20 (2H, br), 8.20-8.60 (2H, br).
   MS (APCI) 126 (M+H⁺).
   Preparation 6

To a solution of 2-(1-methyl-1H-pyrazol-5-yl)ethanamine dihydrochloride (2.97 g) in a mixed solvent of tetrahydrofuran (30 ml) and N,N-dimethylformamide (10 ml) were added triethylamine (4.6 ml) and1-{[(tert-butoxycarbonyl)amino]-[(tert-butoxycarbonyl)imino]methyl}-1H-pyrazole (5.59 g) successively at room temperature. The mixture was stirred at room temperature for 4 days. To the reaction mixture was added water to quench the reaction. The mixture was extracted with ethyl acetate. The organic layer was washed with water and brine. The extract was dried over anhydrous magnesium sulfate, filtered and concentrated in vacuo. The residue was purified by silica gel column chromatography to give 1-{[(tert-butoxycarbonyl)amino]-[(tert-butoxycarbonyl)imino]methyl}-amino-2-(1-methyl-1H-pyrazol-5-yl) ethane (4.58 g) as a solid.

IR (KBr) 3323, 1741, 1652, 1626, 1144, 808, 771, 748 cm⁻¹.

¹H-NMR (DMSO-d₆) δ 1.40 (9H, s), 1.47 (9H, s), 2.86 (2H, t, J=7.3 Hz), 3.53 (2H, dt, J=5.7, 7.3 Hz), 3.77 (3H, s), 6.06 (1H, d, J=1.8 Hz), 7.29 (1H, d, J=1.8 Hz), 8.46 (1H, t, J=5.7 Hz), 11.47 (1H, s).

MS (ESI) 390.3 (M+Na⁺).

Preparation 7

To a solution of benzhydryl 7β-tert-butoxycarbonylamino-3-chloromethyl-3-cephem-4-carboxylate (2.15 g) in N,N-dimethylformamide (6.6 ml) was added sodium iodide (625 mg), and the mixture was stirred at room temperature for 30 minutes. To the reaction mixture was added 1-{[(tert-butoxycarbonyl)-amino]-[(tert-butoxycarbonyl)imino]methyl}amino-2-(1-methyl-1H-pyrazol-5-yl)ethane (4.60 g). The whole mixture was stirred at room temperature for 63 hours and poured into a mixture of ethyl acetate and water. The aqueous layer was separated, and the organic layer was washed with brine, dried over sodium sulfate and filtered. The filtrate was concentrated to about 20 ml in vacuo. The concentrate was poured into diisopropyl ether (200 ml), and the resulting precipitate was collected by filtration and dried in vacuo. To a solution of the resulting solid in methylene chloride (14 ml) were added anisole (2.9 ml) and trifluoroacetic acid (5.8 ml). The resulting solution was stirred at room temperature for 2 hours and poured into diisopropyl ether (200 ml). The resulting precipitate was collected by filtration and dried in vacuo to give crude 7β-amino-3-{[3-(2-{[amino(imino)methyl]amino}ethyl)-2-methyl-1-pyrazolio]methyl}-3-cephem-4-carboxylate bis(trifluoroacetate) (1.46 g). This product was used in the next step without further purification.

### EXAMPLE 2

To a solution of crude 7β-amino-3-{[3-(2-[amino(imino)methyl]amino)ethyl)-2-methyl-1-pyrazolio]methyl}-3-cephem-4-carboxylate bis(trifluoroacetate) (1.65 g) and N-(trimethylsilyl)acetamide (3.15 g) in a mixed solvent of N,N-dimethylformamide (7 ml) and tetrahydrofuran (22 ml) was added (Z)-2-(5-amino-1,2,4-thiadiazol-3-yl)-2-(1-carboxy-1-methylethoxyimino)acetic acid methanesulfonyl ester (1.02 g) under ice-cooling. The solution was stirred at the same temperature for 3 hours. The reaction mixture was poured into a mixed solvent of ethyl acetate (100 ml) and diisopropyl ether (100 ml), and the mixture was stirred for 30 minutes. The resulting precipitate was collected by filtration, washed with ethyl acetate and dried in vacuo to give a solid, which was purified by preparative HPLC utilizing ODS column. The eluate containing a desired product was concentrated to about 30 ml in vacuo. The concentrate was adjusted to about pH 3 by addition of concentrated hydrochloric acid and chromatographed on Diaion® HP-20 eluting with 30% aqueous 2-propanol. The eluate was concentrated to about 30 ml in vacuo and lyophilized to give 3-{[3-(2-{[amino(imino)methyl]amino}-ethyl)-2-methyl-1-pyrazolio]methyl}-7β-({(2Z)-2-(5-amino-1,2,4-thiadiazol-3-yl)-2-[(1-carboxy-1-methylethoxy)imino]ethanoyl}amino)-3-cephem-4-carboxylate (55 mg).

IR (KBr) 1772, 1664, 1400, 1362, 1066, 997 cm⁻¹.

¹H-NMR (D₂O) δ 1.52 (3H, s), 1.54 (3H, s), 3.16 (2H, t, J=6.6 Hz), 3.21 and 3.48 (2H, ABq, J=17.7 Hz), 3.62 (2H, t, J=6.6 Hz), 4.00 (3H, s), 5.27 (1H, d, J=4.8 Hz), 5.30 and 5.45 (2H, ABq, J=15.5 Hz), 5.87 (1H, d, J=4.8 Hz), 6.74 (1H, d, J=3.1 Hz), 8.20 (1H, d, J=3.1 Hz).
   MS (ESI) 658.2 (M+Na⁺).
   Preparation 8

Ethyl (2E)-3-(1-methyl-1H-pyrazol-4-yl)-2-propenoate

The title compound was obtained from 1-methyl-1H-pyrazole-4-carbaldehyde in the same manner as in Preparation 1 as an oil.

IR (neat) 2983, 2941, 1701, 1639, 1263, 1167, 1039, 1024, 852 cm⁻¹.

¹H-NMR (DMSO-d₆) δ 1.23 (3H, t, J=7.1 Hz), 3.83 (3H, s), 4.14 (2H, q, J=7.1 Hz), 6.28 (1H, d, J=16.0 Hz) 7.52 (1H, d, J=16.0 Hz), 7.87 (1H, s), 8.10 (1H, s).

MS (APCI) 181 (M+H⁺).

Preparation 9

Ethyl 3-(1-methyl-1H-pyrazol-4-yl)propanoate

The title compound was obtained from ethyl (2E)-3-(1-methyl-1H-pyrazol-4-yl)-2-propenoate in the same manner as in Preparation 2 as a colorless oil.

IR (neat) 2983, 2937, 1734, 1541, 118.6, 1028 cm⁻¹.

¹H-NMR (DMSO-d₆) δ 1.16 (3H, t, J=7.1 Hz), 2.51 (2H, d, J=6.8 Hz), 2.64 (2H, t, J=6.8 Hz), 3.75 (3H, s), 4.05 (2H, q, J=7.1 Hz), 7.22. (H, s), 7.45(1H, s).

MS (APCI) 183 (M+H⁺).

Preparation 10

3-(1-Methyl-1H-pyrazol-4-yl)propanoic acid

The title compound was obtained from ethyl 3-(1-methyl-1H-pyrazol-4-yl)propanoate in the same manner as in Preparation 3 as a colorless solid.

IR (KBr) 1695, 1335, 1279, 1209 1014, 1003, 856 cm⁻¹.

¹H-NMR (DMSO-d₆) δ 2.43 (2H, d, J=7.1 Hz),2.61 (2H, t, J=7.1 Hz), 3.75 (3H, s), 7.23 (1H, s), 7.44 (1H, s), 11.40-12.65 (1H, br).

Preparation 11

To a solution of 3-(1-methyl-1H-pyrazol-4-yl)propanoic acid (11.56 g) in toluene (230 ml) were added benzyl alcohol (15.5 ml), diphenylphosphoryl azide (22.7 g) and triethylamine (11.5 ml). The mixture was refluxed for 8 hours. After cooling to room temperature, ethyl acetate was added to the reaction mixture. The mixture was washed with saturated aqueous sodium hydrogen carbonate, water and brine. The extract was dried over anhydrous magnesium sulfate, filtered and concentrated in vacuo. The residue was purified by silica gel column chromatography to give benzyl 2-(1-methyl-1H-purazol-4-yl)ethylcarbamate (15.05 g) as an oil.

IR (KBr) 2939, 1714, 1701, 1252, 1136, 991 cm⁻¹.

¹H-NMR (DMSO-d₆) δ 2.52 (2H, t-like), 3.13 (2H, dd-like), 3.75 (3H, s), 5.01 (2H, s), 7.23 (1H, s), 7.30-7.41 (6H, m), 7.46 (1H, s).

MS (APCI) 260.07 (M+H⁺).

Preparation 12

A solution of benzyl 2-(1-methyl-1H-pyrazol-4-yl)ethylcarbamate (15.0 g) in methanol (150 ml) was treated with 10% palladium on carbon (3.-75 g) under a hydrogen atmosphere at room temperature for 1 hour. After the catalyst was filtered off, the filtrate was concentrated in vacuo to give 2-(1-methyl-1H-pyrazol-4-yl)ethylamine (7.36 g) as a waxy solid. This product was used in the next step without further purification.

Preparation 13

2-(1-Methyl-1H pyrazol-4-yl)ethylamine (3.73 g) in ethyl formate (75 ml) was refluxed overnight. After cooling to room temperature, the solvent was evaporated in vacuo to give N-[2-(1-methyl-1H-pyrazol-4-yl)ethyl]formamide (7.36 g) as an oil.

IR (neat) 3053, 2939, 2870, 1678, 1394, 1165, 991 cm⁻¹.

¹H-NMR (DMSO-d₆) δ 2.53 (2H, t, J=7.1 Hz), 3.22.(2H, dd, J=7.4, 7.1 Hz), 3.76 (3H, s), 7.26 (1H, s); 7.49 (1H, s), 7.90-8.15 (1H, br), 7.99 (1H, s).

MS (ESI) 176.2 (M+Na⁺).

Preparation 14

7β-Amino-3-({4-[2-(formylamino)ethyl]-2-methyl-1-pyrazolio}methyl)-3-cephem-4-carboxylate trifluoroacetate

The title compound was obtained from benzhydryl 7β-tert-butoxycarbonylamino-3-chloromethyl-3-cephem-4-carboxylate and N-[2-(1-methyl-1H-pyrazol-4-yl)ethyl]formamide in the same manner as in Preparation 7. This product was used in the next step without further purification.

### EXAMPLE 3

To a solution of 7β-amino-3-({4-[2-(formylamino)ethyl]-2-methyl-1-pyrazolio}methyl)-3-cephem-4-carboxylate trifluoroacetate (3.53 g) and N-(trimethylsilyl)acetamide (9.66 g) in a mixed solvent of N,N-dimethylformamide (7 ml) and tetrahydrofuran (53 ml) was added (Z)-2-(5-amino-1,2,4-thiadiazol-3-yl)-2-(1-carboxy-1-methylethoxyimino)acetyl chloride (1.02 g) under ice-cooling. The mixture was stirred at the same temperature for 2 hours. The reaction mixture was poured into a mixed solvent of ethyl acetate (100 ml) and diisopropyl ether (100 ml), and the mixture was stirred for 30 minutes. The resulting precipitate was collected by filtration, washed with ethyl acetate and dried in vacuo to give a solid. To a solution of the resulting solid in methylene chloride (25 ml) were added anisole (5 ml) and trifluoroacetic acid (10 ml). The resulting solution was stirred at room temperature for 2 hours and poured into diisopropyl ether (200 ml). The resulting precipitate was collected by filtration and dried in vacuo to give crude solid. The obtained solid was dissolved in methanol (33 ml), and concentrated hydrochloric acid (6.0 ml) was added to the solution. The mixture was stirred for 8.5 hours and poured into ethyl acetate (100 ml). The resulting precipitate was collected by filtration, washed with ethyl acetate and dried in vacuo to give a solid which was purified by preparative HPLC utilizing ODS column. The eluate containing a desired product was concentrated to about 30 ml in vacuo. The concentrate was adjusted to about pH 3 by addition of concentrated hydrochloric acid and chromatographed on Diaion® HP-20 eluting with 30% aqueous 2-propanol. The eluate was concentrated to about 30 ml in vacuo and lyophilized to give 3-{[4-(2-aminoethyl)-2-methyl-1-pyrazolio]methyl}-7β-({(2Z)-2-(5-amino-1,2,4-thiadiazol-3-yl)-2-[(1-carboxy-1-methylethoxy)imino]ethanoyl}amino)-3-cephem-4-carboxylate (118 mg).

IR (KBr) 1774, 1603, 1525, 1398, 1159 cm⁻¹.

¹H-NMR (D₂O) δ 1.53 (3H, s); 1.54 (3H, s), 2.95 (2H, t, J=7.4 Hz), 3.25 (2H, t, J=7.4 Hz), 3.26 and 3.60 (2H, ABq, J=17.9 Hz), 4.09 (3H, s), 5.22 and 5.35 (2H, ABq, J=15.3 Hz), 5.29 (1H, d, J=4.9 Hz)., 5.87 (1H, d, J=4.9 Hz), 8.19 (2H, s).

MS (ESI, negative) 592.2 (M−H⁺).

Preparation 15

To a solution of 2-(1H-pyrazol-1-yl)ethanol (18.5 g), phthalimide (24.2 g) and triphenylphosphine (47.7 g) in tetrahydrofuran (650 ml) was added diisopropyl azodicarboxylate (36.8 g) under ice-cooling, and the mixture was stirred at room temperature for 2 hours. The solvent was evaporated. To the residue was added ethyl acetate and washed with 2N aqueous hydrochloric acid solution (×5). The combined aqueous layers were adjusted to pH 7 with 10% aqueous sodium carbonate solution and extracted with ethyl acetate. The organic layer was dried over anhydrous magnesium sulfate, filtered and concentrated in vacuo. The residue was purified by silica gel column chromatography to give 1-(2-phthalimidoethyl)-1H-pyrazole (20.02 g) as a solid.

¹H-NMR (DMSO-d₆) δ 3.93 (2H, t, J=5.9 Hz), 4.39 (2H, t, J=5.9 Hz), 6.16 (1H, dd, J=2.1, 1.4 Hz), 7.31 (1H, d, J=1.4 Hz), 7.71 (1H, d, J=2.1 Hz), 7.79-7.88 (4H, m).

MS (ESI) 264.3 (M+Na⁺).

Preparation 16

To a solution of 1-(2-phthalimidoethyl)-1H-pyrazole (6.86 g) in ethanol (68 ml) was added hydrazine monohydrate (1.40 ml), and the mixture was refluxed for 45 minutes. After cooling to 0° C., insoluble materials were filtered off and washed with ethanol. The filtrate was concentrated in vacuo. The residue was purified by silica gel column chromatography to give 2-(1H-pyrazol-1-yl)ethylamine (2.65.g) as an oil.

¹H-NMR (DMSO-d₆) δ 2.88 (2H, t, J=6.3 Hz), 4.06 (2H, t, J=6.3 Hz), 6.22 (1H, dd, J=2.1, 1.4 Hz), 7.42 (1H, d, J=1.4 Hz), 7.69 (1H, d, J=2.1 Hz).

Preparation 17

N-[2-(1H-Pyrazol-1-yl)ethyl]formamide

The title compound was obtained from 2-(1H-pyrazol-1-yl)ethylamine in the same manner as in Preparation 13.

IR (neat) 1678, 1541, 1514, 1396, 758, 619, 525 cm⁻¹.

¹H-NMR (DMSO-d₆) δ 3.47 (2H, dd, J=12.2, 6.3 Hz), 4.17(2H, t, J=6.3 Hz), 6.23 (1H, dd, J=2.3, 1.7 Hz), 7.45 (1H, d, J=1.7 Hz), 7.69 (1H, d, J=2.3 Hz), 8.07 (1H, br-s).

MS (API) 140 (M+H⁺).

Preparation 18

7β-Amino-3-({2-[2-(formylamino)ethyl]-1-pyrazolio}methyl)-3-cephem-4-carboxylate trifluoroacetate

The title compound was obtained from benzhydryl 7β-tert-butoxycarbonylamino-3-chloromethyl-3-cephem-4-carboxylate and N-[2-(1H-pyrazol-1-yl)ethyl]formamide in the same manner as in Preparation 7. This product was used in the next step without further purification.

Preparation 19

To a solution of crude 7β-amino-3-({2-[2-(formylamino)ethyl]-1-pyrazolio}methyl)-3-cephem-4-carboxylate trifluoroacetate (2.06 g) and N-trimethylsilylacetamide (5.81 g) in a mixed solvent of N,N-dimethylformamide (10 ml) and tetrahydrofuran (30 ml) was added (Z)-2-(5-amino-1,2,4-thiadiazol-3-yl)-2-(1-carboxy-1-methylethoxyimino)acetyl chloride hydrochloride (1.87 g) under ice-cooling. The solution was stirred at the same temperature for 2.5 hours. The reaction mixture was poured into a mixed solvent of ethyl acetate (100 ml) and diisopropyl ether (100 ml), and the mixture was stirred for 30 minutes. The resulting precipitate was collected by filtration, washed with ethyl acetate, and dried in vacuo to give a solid. To a solution of the resulting solid in methanol (17 ml) was added concentrated hydrochloric acid (3.7 ml). The resulting solution was stirred at room temperature for 4.5 hours and poured into ethyl acetate (200 ml). The resulting precipitate was collected by filtration, washed with ethyl acetate, and dried in vacuo to give a solid, which was purified by preparative HPLC utilizing ODS column. The eluate containing a desired product was concentrated to about 30 ml in vacuo. The concentrate was adjusted to about pH 3 by addition of concentrated hydrochloric acid and chromatographed on Diaion® HP-20 eluting with 30% aqueous 2-propanol. The eluate was concentrated to about 30 ml in vacuo and lyophilized to give 3-{[2-(2-aminoethyl)-1-pyrazolio]methyl}-7β-({(2Z)-2-(5-amino-1,2,4-thiadiazol-3-yl) -2-[(1-carboxy-1-methylethoxy)imino]ethanoyl)amino)-3-cephem-4-carboxylate (80 mg).

¹H-NMR (D₂O) δ 1.53 (6H, s), 3.11 and 3.61 (2H, ABq, J=17.9 Hz), 3.52 (2H, t, J=6.7 Hz), 5.02 (2H, t, J=6.7 Hz), 5.31 (1H, d, J=4.9 Hz), 5.39 and 5.60 (2H, ABq, J=15.3 Hz), 5.86 (1H, d, J=4.9 Hz), 6.91 (1H, dd, J=3.0, 3.0 Hz), 8.40 (1H, d, J=3.0 Hz), 8.42 (1H, d, J=3.0 Hz).

MS (ESI) 580.2 (M+H⁺).

Preparation 20

To a solution of 1-methyl-4-nitro-1H-pyrazole (12.06 g) in a mixed solvent of ethanol (120 ml) and tetrahydrofuran (120 ml) were added 10% palladium on carbon (6.6 g) and di-tert-butyl dicarbonate (22.8 g). The mixture was stirred under 3.0 atm. hydrogen atmosphere for 1.5 hours at ambient temperature. After the catalyst was filtered off, the filtrate was concentrated in vacuo. The residual solid was triturated with hexane, filtered, washed with hexane, and dried under vacuum to give tert-butyl 1-methyl-1H-pyrazol-4-ylcarbamate (18.9 g) as a solid.

¹H-NMR (DMSO-d₆) δ 1.43 (9H, s), 3.74 (3H, s), 7.24 (1H, s), 7.60 (1H, s), 9.11 (1H, br-s).

Preparation 21

A solution of tert-butyl 1-methyl-1H-pyrazol-4-ylcarbamate (986 mg) in tetrahydrofuran (10 ml) was cooled to −70° C. To the solution was added dropwise 1.6M n-butyllithium in hexane (6.6 ml) at a temperature below −50° C. The mixture was stirred for 1 hour at −60° C. To the reaction mixture was added N,N-dimethylformamide (0.426 ml) dropwise, and the mixture was stirred for 1 hour at −60° C. To the resulting mixture were added 10% aqueous potassium hydrogen sulfate solution, and the mixture was extracted with ethyl acetate. The organic layer was washed with brine, dried over anhydrous magnesium sulfate, filtered and concentrated in vacuo. The residue was purified by silica gel column chromatography to give tert-butyl 5-formyl-1-methyl-1H-pyrazol-4-ylcarbamate (480 mg) as a colorless solid.

IR (KBr) 3350, 1714, 1668, 1574, 1471, 1417, 1240, 1155, 1001, 835 cm⁻¹.

¹H-NMR (DMSO-d₆) δ 1.48 (9H, s); 4.01 (3H, s), 7.73 (1H, s), 9.36 (1H, br-s), 9.95 (1H, s).

MS (APCI, negative) 223.67 (M−H⁺).

Preparation 22

Ethyl (2E)-3-(4-[(tert-butoxycarbonyl)amino]-1-methyl-1H-pyrazol-5-yl}-2-propenoate

The title compound was obtained from tert-butyl 5-formyl-1-methyl-1H-pyrazol-4-ylcarbamate in the same manner as in Preparation 1 as a solid.

IR(neat) 3327, 1713, 1695, 1639, 1576, 1506, 1273, 1252, 1163 cm⁻¹.

¹H-NMR (DMSO-d₆) δ 1.26 (3H, t, J=7.1 Hz), 1.44 (9H, s), 3.90 (3H, s), 4.20 (2H, q, J=7.1 Hz), 6.37 (1H, d, J=16.3 Hz), 7.47 (1H, s), 7.59 (1H, d, J=16.3 Hz), 8.90 (1H, br-s).

MS (ESI) 318.3 (M+Na⁺).

Preparation 23

Ethyl 3-{4-[(tert-butoxycarbonyl)amino]-1-methyl-1H-pyrazol-5-yl}propanoate

The title compound was obtained from ethyl (2E)-3-{4-[(tert-butoxycarbonyl)amino]-1-methyl-1H-pyrazol-5-yl}2-propenoate in the same manner as in Preparation-2 as a solid.

IR (KBr) 1736, 1714, 1701, 1047, 1020 cm⁻¹.

¹H-NMR (DMSO-d₆) δ 1.05 (3H, t, J=7.0 Hz), 1.42 (9H, s) 2.49-2.56 (2H, t-like), 2.83(2H, t-like), 3.70 (3H, s), 4.06 (2H, q, J=7.0 Hz), 7.30 (1H, s), 8.40 (1H,-br-s).

Preparation 24

3-{4-[(tert-Butoxycarbonyl)amino]-1-methyl-1H-pyrazol-5-yl}propanoic acid

The title compound was obtained from ethyl 3-{4-[(tert-butoxycarbonyl)amino]-1-methyl-1H-pyrazol-5-yl}propanoate in the same manner as in Preparation 3 as a colorless solid.

IR (KBr) 3307, 1726, 1693, 1610, 1369, 1049, 1024 cm⁻¹.

¹H-NMR (DMSO-d₆) δ 1.42 (9H, s), 2.44 (2H, t-like), 2.79 (2H, t-like), 3.70 (3H, s), 7.30 (1H, s), 8.40 (1H, br-s), 12.25 (1H, br-s).

MS (APCI) 269.73 (M+H⁺).

Preparation 25

To a solution of 3-(4-[(tert-butoxycarbonyl)amino]-1-methyl-1H-pyrazol-5-yl)propanoic acid (8.3 g) in toluene (170 ml) were added diphenylphosphoryl azide (10.2 g) and triethylamine (5.15 ml). The mixture was refluxed for 3.5 hours. After cooling to room temperature, the solvent was evaporated. To the residue were added tetrahydrofuran (60 ml) and 1.0N aqueous sodium hydroxide solution (25 ml). The mixture was refluxed overnight. After cooling to room temperature, the pH of the aqueous phase was adjusted to pH 8.5 with 1.0N hydrochloric acid. To the mixture was added di-tert-butyl dicarbonate (22.8 g), and the mixture was stirred for 1 hour. The reaction mixture was extracted with ethyl acetate. The organic layer was washed with water and brine. The extract was dried over anhydrous magnesium sulfate, filtered and concentrated in vacuo. The residue was purified by silica gel column chromatography to give tert-butyl 5-{2-[(tert-butoxycarbonyl)amino]ethyl}-1-methyl-1H-pyrazol-4-ylcarbamate (1.90 g) as a solid.

¹H-NMR (DMSO-d₆) δ 1.38 (9H, s), 1.43 (9H, s), 2.70 (2H, t-like), 3.04(2H, dd-like), 3.70 (3H, s), 6.89 (2H, t-like), 7.34 (1H, s), 8.39 (1H, br-s).

MS (ESI) 341.11 (M+H⁺).

Preparation 26

To a solution of 4-methoxybenzyl 7β-[(Z)-2-(amino-1,2,4-thiadiazol-3-yl)-2-(1-tert-butoxycarbonyl-1-methylethoxyimino)acetamido]-3-chloromethyl-3-cephem-4-carboxylate (3.22 g) in N,N-dimethylformamide (10 ml) was added 1,3-bis(trimethylsilyl)urea (4.83 g). After stirring at room temperature for 0.5 hours, to the mixture was added potassium iodide (863 mg), and the mixture was stirred for further 1 hour. 5-{2-[(tert-butoxycarbonyl)amino]ethyl}-1-methyl-1H-pyrazol-4-ylcarbamate (2.01 g) was added to the mixture, and stirring was continued for 24 hours at 37° C. The resulting mixture was poured into water and extracted with ethyl acetate. The organic layer was washed with water, 10% aqueous sodium thiosulfate solution, brine and 10% aqueous sodium trifluoroacetate, dried over magnesium sulfate, filtered and evaporated in vacuo. The residue was dissolved in small amount of ethyl acetate, and the solution was added to diisopropyl ether dropwise. The precipitate was collected by filtration. The filter cake was washed with diisopropyl ether and dried under vacuum.

To absolution of the resulting solid in methylene chloride (7.6 ml) were added anisole (3.8 ml) and trifluoroacetic acid (11.4 ml). The resulting solution was stirred for 4 hours at room temperature and poured into diisopropyl ether. The resulting precipitate was collected by filtration and dried in vacuo.

The crude product was purified by preparative HPLC utilizing ODS column. The first eluate containing a desired product was concentrated to about 30 ml in vacuo. The concentrate was adjusted to about pH 3 by addition of concentrated hydrochloric acid and chromatographed on Diaion® HP-20 eluting with 30% aqueous 2-propanol. The eluate was concentrated to about 30 ml in vacuo, added with 2.0M aqueous sulfuric acid (0.245 ml) and lyophilized to give 3-{[4-amino-3-(2-aminoethyl)-2-methyl-1-pyrazolio]methyl}-7β-({(2Z)-2-(5-amino-1,2,4-thiadiazol-3-yl)-2-[(1-carboxy-1-methylethoxy)imino]ethanoyl}amino)-3-cephem-4-carboxylic acid hydrogen sulfate (415 mg).

IR (KBr) 1776, 1714, 1674, 1628, 1527, 1155, 1111 cm⁻¹.

¹H-NMR (D₂O) δ 1.61(6H, s), 3.24 and 3.53 (2H, ABq, J=17.8 Hz), 3.24-3.35 (4H, m), 3.97 (3H, s), 5.21 and 5.39 (2H, ABq, J=15.5 Hz), 5.25 (1H, d, J=4.7 Hz), 5.89 (1H, d, J=4.7 Hz), 7.87 (1H, s).

MS (ESI) 609.2 (M+H⁺).

This application is based on application. No. 2003902380 filed in Australia on May 16, 2003, the content of which is incorporated hereinto by reference.

1. A compound of the formula [I]: 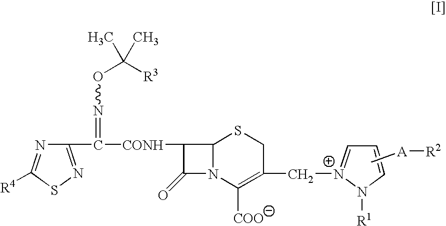
wherein
R¹ is lower alkyl which may have suitable substituent(s),
R² is amino, protected amino or guanidino,
R³ is carboxy or protected carboxy,
R⁴ is amino or protected amino, and
A is lower alkylene,
or a pharmaceutically acceptable salt thereof.

2. A process for preparing a compound of the formula [I]: 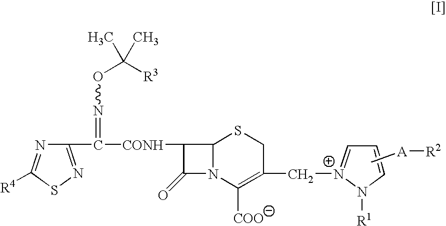
wherein
R¹ is lower alkyl which may have suitable substituent(s),
R² is amino, protected amino or guanidino,
R³ is carboxy or protected carboxy,
R⁴ is amino or protected amino, and
A is lower alkylene,
or a salt thereof, which comprises
(1) reacting a compound of the formula [II]: 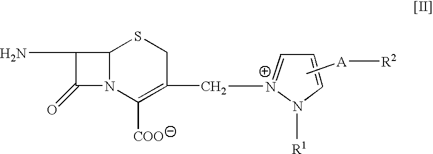
wherein R¹, R² and A are each as defined above, or its reactive derivative at the amino group, or a salt thereof with a compound of the formula [III]: 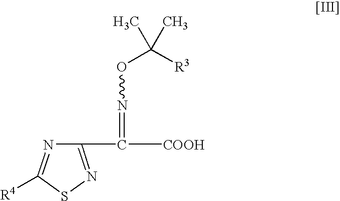
wherein R³ and R⁴ are each as defined above, or its reactive derivative at the carboxy group, or a salt thereof to give a compound of the formula [I]: 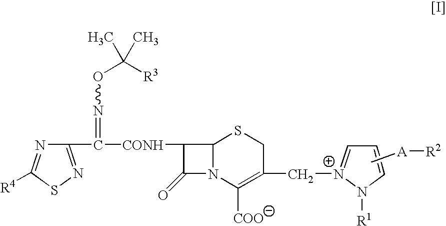
wherein R¹, R², R³, R⁴ and A are each as defined above, or a salt thereof, or
(2) reacting a compound of the formula [VI]: 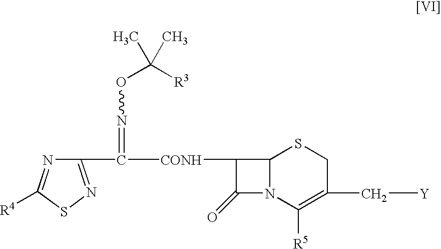
wherein R³ and R⁴ are each as defined above, R⁵ is protected carboxy, and Y is a leaving group, or a salt thereof with a compound of the formula [VII]: 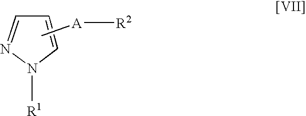
wherein R¹, R² and A are each as defined above, or a salt thereof to give a compound of the formula [VIII]: 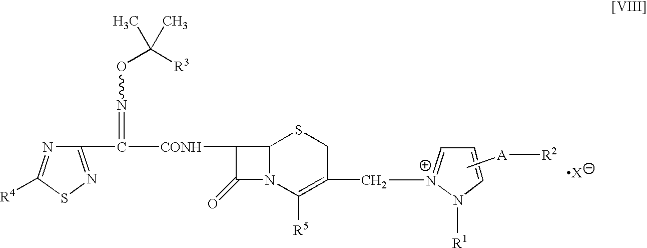
wherein R¹, R², R³, R⁴, R⁵ and A are each as defined above, and X^{\mathring{-}} is an anion, or a salt thereof, and subjecting the compound of the formula [VIII] or a salt thereof to elimination reaction of the carboxy protecting group, to give a compound of the formula [I]: 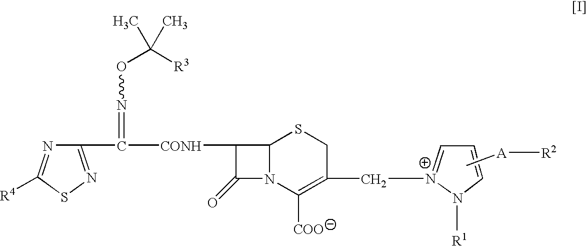
wherein R¹, R², R³, R⁴ and A are each as defined above, or a salt thereof.

3. A pharmaceutical composition comprising a compound of claim 1 or a pharmaceutically acceptable salt thereof in admixture with a pharmaceutically acceptable carrier.

4. A compound of claim 1 or a pharmaceutically acceptable salt thereof for use as a medicament.

5. A compound of claim 1 or a pharmaceutically acceptable salt thereof for use as an antimicrobial agent.

6. Use of a compound of claim 1 or a pharmaceutically acceptable salt thereof for manufacture of a medicament for treating infectious diseases.

7. A method for the treatment of infectious diseases which comprising administering a compound of claim 1 or a pharmaceutically acceptable salt thereof to human or animals.
